(19) United States
(12) Patent Application Publication
(10) Pub. No.: US 2013/0052207 A1
(43) Pub. Date: Feb. 28, 2013

(54) Prognostic Method for Pulmonary Adenocarcinoma, Pulmonary Adenocarcinoma Detection Kit, and Pharmaceutical Composition for Treating Pulmonary Adenocarcinoma

(71) Applicants: Kazufumi Honda, Tokyo (JP); Tesshi Yamada, Tokyo (JP); Rintaro Noro, Tokyo (JP); Setsuo Hirohashi, Tokyo (JP)

(72) Inventors: Kazufumi Honda, Tokyo (JP); Tesshi Yamada, Tokyo (JP); Rintaro Noro, Tokyo (JP); Setsuo Hirohashi, Tokyo (JP)

(73) Assignee: JAPAN HEALTH SCIENCES FOUNDATION, Tokyo (JP)

(21) Appl. No.: 13/636,323

(22) PCT Filed: Mar. 29, 2011
(86) PCT No.: PCT/JP2011/057866

§ 371 Date: Sep. 20, 2012

(30) Foreign Application Priority Data
Mar. 30, 2010 (JP) 2010-078772

(51) Int. Cl. G01N 21/64; G01N 33/574; C07H 21/04; A61P 35/00; A61K 39/395; A61K 31/7088; A61K 31/713; G01N 33/577; C07K 16/18

(52) U.S. Cl. 424/174.1; 435/6.11; 435/7.1; 435/7.92; 536/24.31; 530/389.7; 514/44 A

(57) ABSTRACT

A method of detecting a poor-prognosis stage I lung adenocarcinoma in a mammal, comprising a step of measuring the copy number of ACTN4 gene in the cells of the stage I lung adenocarcinoma tissue obtained from the mammal, wherein, when the copy number of the gene exceeds 4, the stage I lung adenocarcinoma in the mammal is predicted to be a poor-prognosis stage I lung adenocarcinoma.

### TECHNICAL FIELD

The present invention relates to a prognostic method for pulmonary adenocarcinoma, a pulmonary adenocarcinoma detection kit, and a pharmaceutical composition for treating pulmonary adenocarcinoma.

### BACKGROUND ART

Clinical stage I lung adenocarcinoma is an early lesion that has not yet metastasized to sites other than the primary focus. According to the current classification, stage I lung adenocarcinoma is further classified into stage IA (the diameter of tumor of less than 3 cm) and stage IB (the diameter of tumor of 3 cm or larger). According to Kato et al., the postoperative five-year survival rate of clinical stage I lung adenocarcinoma is 85%, and it has been verified that postoperative chemotherapy (oral administration of UFT for two years) significantly contributes to improving the prognosis in stage I lung adenocarcinoma (Non-Patent Document 1: Kato H, Ichinose Y, Ohta M, Hata E, Tsubota N, Tada H, Watanabe Y, Wada H, Tsuboi M, Hamajima N, Ohta M; Japan Lung Cancer Research Group on Postsurgical Adjuvant Chemotherapy. A randomized trial of adjuvant chemotherapy with uracil-tegafur for adenocarcinoma of the lung; N. Engl. J. Med., 2004 Apr. 22; 350 (17): 1713-21). Currently, it is recommended that postoperative chemotherapy be given to patients with stage I lung adenocarcinoma; however, not all the patients with stage I lung adenocarcinoma are benefited from the postoperative chemotherapy. Accordingly, it is considered that if postoperative prognosis is predicted, a risk that unnecessary chemotherapy is given to a group of patients with favorable prognosis can be reduced.

A molecule, α-actinin-4, has been isolated as an actin-binding protein involved in the infiltration and metastasis of a cancer cell (Non-Patent Document 2: Honda et al., J. Cell Biol. 1998 Mar. 23; 140 (6): 1383-93). Further, it has been reported that an expression of α-actinin-4 protein is involved in the cell movement and infiltration property (Non-Patent Document 3: Honda et al., Gastroenterology. 2005 January; 128 (1): 51-62).

### PRIOR ART LITERATURE

### Non-Patent Literature

Non-Patent Document 1: Kato H. et al., N. Engl. J. Med., 2004 Apr. 22; 350(17): 1713-21
Non-Patent Document 2: Honda et al., J. Cell Biol. 1998 Mar. 23; 140 (6): 1383-93
Non-Patent Document 3: Honda et al., Gastroenterology. 2005 January; 128 (1): 51-62

### SUMMARY OF INVENTION

### Problems to be Solved by the Invention

The present inventors have found that patients with an enhanced α-actinin-4 (ACTN4) protein expression in a stage I lung adenocarcinoma tissue exhibit a shorter overall survival (OS) than do patients without enhanced ACTN4 expression with statistically significant difference. The present inventors have also found that patients in a group having an increased copy number of α-actinin-4 protein (ACTN4) gene in the genome of a stage I lung adenocarcinoma tissue cell exhibit a shorter OS than do patients in a group not having an increased copy number of the gene. The present inventors also found that OS of patients with stage I lung adenocarcinoma can be more precisely predicted by a combination of a copy number of ACTN4 gene and an expression level of α-actinin-4 protein compared to an independent analysis conducted with respect to each of the copy number of ACTN4 gene and the expression level of α-actinin-4 protein. The present invention has been accomplished based on the findings as described above.

Accordingly, an object of the present invention is to provide a prognostic method for pulmonary adenocarcinoma, a pulmonary adenocarcinoma detection kit, and a pharmaceutical composition for treating pulmonary adenocarcinoma.

### Means for Solving the Problem

A diagnostic method according to a first aspect of the present invention is a method for detecting poor-prognosis stage I lung adenocarcinoma in a mammal, including a step of measuring the copy number of α-actinin-4 gene in a stage I lung adenocarcinoma tissue cell obtained from the aforementioned mammal. According to the present method, when the copy number of the aforementioned gene exceeds four, it is indicated that the stage I lung adenocarcinoma developed in the mammal is poor-prognosis stage I lung adenocarcinoma.

A kit according to a second aspect of the present invention is a kit for detecting a poor-prognosis stage I lung adenocarcinoma in a mammal, comprising a reagent that is available for determining the copy number of ACTN4 gene in a cell.

A diagnostic method according to a third aspect of the present invention is a method for detecting poor-prognosis stage I lung adenocarcinoma in a mammal, including the steps of (a) measuring an expression level of α-actinin-4 protein in a stage I lung adenocarcinoma tissue or cell obtained from the aforementioned mammal and (b) comparing the expression level obtained in the step (a) with an expression level of α-actinin-4 gene in a corresponding normal tissue or cell. According to the present method, when an expression of the aforementioned protein or gene in the aforementioned stage I lung adenocarcinoma tissue or cell is enhanced, it is indicated that the stage I lung adenocarcinoma developed in the mammal is poor-prognosis stage I lung adenocarcinoma.

A kit according to a fourth aspect of the present invention is a kit for detecting a poor-prognosis stage I lung adenocarcinoma in a mammal, comprising a reagent that can measure the expression level of ACTN4 gene in the tissue or cell.

A pharmaceutical composition according to a fifth aspect of the present invention is a pharmaceutical composition for treating stage I lung adenocarcinoma, containing an α-actinin-4 inhibitor and a pharmaceutically acceptable carrier.

A kit according to a sixth aspect of the present invention is a kit for measuring the copy number of ACTN4 gene in human cells, comprising a probe specific for the gene on genome that can be used in a FISH method, wherein the specific probe has the nucleotide sequence of the region from 43.81 Mb to 44.02 Mb on human chromosome 19.

### Effect of the Invention

According to the present invention, prediction of prognosis of stage I lung adenocarcinoma becomes possible, and therefore appropriate therapy can be selected. For example, when stage I lung adenocarcinoma is determined as poor-prognosis stage I lung adenocarcinoma according to the present invention, chemotherapy using a medicine such as a combination preparation of tegafur and uracil, and cisplatin is recommended after extirpation of the adenocarcinoma. On the other hand, when stage I lung adenocarcinoma is not determined as poor-prognosis stage I lung adenocarcinoma according to the present invention, unnecessary chemotherapy can be avoided. Furthermore, according to the present invention, means for treating stage I lung adenocarcinoma, for example, poor-prognosis stage I lung adenocarcinoma, is provided.

### BRIEF DESCRIPTION OF DRAWINGS

FIGS. 1(A) and 1(B) are graphs showing survival curves for the two groups, as prepared by the Kaplan-Meier method, positive cases and negative cases of acceleration of α-actinin-4 protein expression in lung adenocarcinoma tissue (stages I and IA) (cohort 1).

FIGS. 2(A) and 2(B) are graphs showing survival curves for two groups, as prepared by Kaplan-Meier method, positive cases and negative cases of acceleration of α-actinin-4 protein expression in lung adenocarcinoma tissue (stages I and IA) (cohort 2).

FIGS. 3(A) and 3(B) are graphs showing survival curves for three groups, stage I lung adenocarcinoma patients, prepared by the Kaplan-Meier method: a group of a serum CEA of 5 ng/mL or less and actinin-4 negative; a group of a serum CEA of more than 5 ng/mL or α-actinin-4 protein positive; and a group of a serum CEA of more than 5 ng/mL and α-actinin-4 protein positive.

FIG. 4 is a graph showing survival curves for the three groups, the stage I lung adenocarcinoma patients as prepared by the Kaplan-Meier method: a group of ACTN4 amplification (−) and α-actinin-4 protein expression negative; a group of ACTN4 gene amplification (−) and α-actinin-4 protein expression positive; and a group of ACTN4 gene amplification (+) and α-actinin-4 protein expression positive.

FIG. 5 contains photo-micrographs showing expression in lung adenocarcinoma cells with immunohistochemical staining FIGS. 6(A) and 6(B) are graphs showing survival curves for the two groups, as prepared by the Kaplan-Meier method, positive cases and negative cases of acceleration of α-actinin-4 protein expression in lung adenocarcinoma tissue (stages Ito III and stage I) (cohort 1 and 2).

FIG. 7 contains photo-micrographs of lung adenocarcinoma cells labeled by the fluorescent in situ hybridization (FISH) method.

FIGS. 8(A) and 8(B) are graphs showing survival curves for the two groups, as prepared by the Kaplan-Meier method, positive cases and negative cases of tissue microarrays in lung adenocarcinoma tissue (stage I).

FIGS. 9(A), 9(B), 9(C) and 9(D) are graphs showing survival curves for the four groups, as prepared by the Kaplan-Meier method, positive cases and negative cases of acceleration of α-actinin-4 protein expression and tissue microarrays in lung adenocarcinoma tissue (stages I and IA) (cohort 1 and 2).

### MODE FOR CARRYING OUT THE INVENTION

Cancer to be treated by the present invention is stage I lung adenocarcinoma. Lung cancer is roughly classified into non-small cell carcinoma and small cell carcinoma. Lung adenocarcinoma is classified into non-small cell carcinoma. Stage I lung adenocarcinoma is the earliest stage of lung adenocarcinoma, which has not yet metastasized to a lymph node and other organs. According to the diagnostic method of the present invention, whether or not stage I lung adenocarcinoma developed in a mammal is poor-prognosis can be determined. Further, a disease to be treated by the present invention is stage I lung adenocarcinoma, preferably poor-prognosis stage I lung adenocarcinoma. In another preferred embodiment, a disease to be treated by the present invention is stage I lung adenocarcinoma exhibiting amplified ACTN4 genes or an enhanced expression of the gene in the carcinoma cells, particularly, stage I lung adenocarcinoma determined as poor-prognosis according to the diagnostic method of the present invention.

In the present specification, the term “poor-prognosis” means that, for example, a five-year survival rate after excision of cancer is less than 90%, preferably less than 85%, more preferably less than 70%, and most preferably less than 60%. Also, “poor-prognosis” in the present specification means that chemotherapy is required after excision of cancer. Therefore, according to another embodiment of the present invention, the diagnostic method of the present invention is a method for detecting stage I lung adenocarcinoma requiring postoperative chemotherapy in a mammal or a method for determining whether or not a patient with stage I lung adenocarcinoma requires postoperative chemotherapy.

The “α-actinin-4” or “ACTN4”, which is used as an index of diagnosis and targeted in the treatment according to the present invention, can be α-actinin-4 (ACTN4 gene product) derived from a mammal subjected to diagnosis or treatment. The ACTN4 gene sequence and the sequence of amino acid encoded by the gene are known in the art, and examples thereof include the sequences of human-derived ACTN4 shown in SEQ ID NOs: 1 and 2 (Ensembl ID:ENSG00000130402 and ENST00000252699). Sequences of human-derived ACTN4 are also registered as NCBI accession numbers: NM_{—}004924 and NP_{—}004915. Further, the gene locus of the ACTN4 gene is also known in the art, for example, the human ACTN4 gene is located on chromosome 19 between base positions 43,830,167 and 43,913,010. With respect also to mammalian animals other than humans, similar genes and proteins corresponding to human-derived ACTN4 are available.

In the present specification, it is verified that amplified ACTN4 genes (an increased copy number in the genome) and an enhanced expression of α-actinin-4 protein, which is a transcription product of the gene, are poor-prognosis factors for stage I lung adenocarcinoma. Further, it is verified that a combination of amplified ACTN4 genes and an enhanced expression of ACTN4 protein are poor-prognosis factors based on which prognosis of stage I lung adenocarcinoma can be more precisely predicted compared to an independent analysis conducted with respect to each one of them. Accordingly, by examining the copy number of ACTN4 gene in a stage I lung adenocarcinoma tissue cell obtained from a subject mammal (humans or mammals other than humans), whether or not the stage I lung adenocarcinoma is poor-prognosis can be diagnosed. Further, by examining an enhanced expression of α-actinin-4 protein in a stage I lung adenocarcinoma tissue or cell obtained from the aforementioned mammal, whether or not the stage I lung adenocarcinoma is poor-prognosis can also be diagnosed.

A stage I lung adenocarcinoma tissue or cell subjected to a diagnosis can be an operative sample extirpated from a patient by surgery or a biopsy sample obtained by endoscopy and the like.

In a diagnostic method according to a first aspect of the present invention, whether or not stage I lung adenocarcinoma is poor-prognosis is determined by examining the copy number of ACTN4 gene in a stage I lung adenocarcinoma tissue cell, whereby poor-prognosis stage I lung adenocarcinoma is detected. The above method includes the step of measuring the copy number of ACTN4 gene in a stage I lung adenocarcinoma tissue cell. When the copy number exceeds 4, stage I lung adenocarcinoma in a subject mammal is determined (diagnosed) as poor-prognosis stage I lung adenocarcinoma.

A publicly known, standardized method in the art can be used to measure the copy number of ACTN4 gene. The ACTN4 gene sequence and the sequence of an amino acid encoded by the gene are as described above, and a person skilled in the art can measure the copy number of ACTN4 gene using a standardized method with reference to these sequences. Examples of a method for measuring the copy number of the gene include, particularly, an assay based on hybridization and amplification.

Examples of the assay based on hybridization include Southern blotting. In this method, genomic DNA is fragmented and separated by electrophoresis, and transferred to a membrane and hybridized with an ACTN4-specific probe. A relative copy number of ACTN4 gene is obtainable by comparing the hybridization signal intensity emitted by the specific probe with the signal intensity emitted by a control probe, which will hybridize with a region in which the copy number of the gene is not increased in the same genome.

According to a preferred embodiment of the present invention, measurement of the copy number of ACTN4 gene is performed by florescence in situ hybridization (FISH), which uses a probe specific to the gene in the genome (refer to Angerer, Meth. Enzymol. 152, 649, 1987). Generally, in situ hybridization is mainly performed as follows; a tissue or cell to be analyzed is immobilized and subjected to pre-hybridization treatment. Then, a probe specific to the nucleic acid of the tissue or cell is hybridized, followed by a wash to remove an unbound specific probe. Subsequently, a hybridized specific probe is detected. The specific probe used in the above-described application is labeled with a fluorescence reporter. The copy number of ACTN4 gene in a cell is obtainable by counting the number of fluorescence in the nucleus of the cell.

According to a particularly preferred embodiment of the present invention, the aforementioned specific probe used for FISH consists of a nucleotide sequence of a region from 43.81 Mb to 44.02 Mb on human chromosome 19. A probe consisting of the nucleotide sequence of the above region is particularly effective in measuring the copy number of ACTN4 gene in the genome, which enables accurate measurement with high sensitivity. Therefore, according to another embodiment of the present invention, a kit for measuring the copy number of α-actinin-4 gene in a human cell is provided. The kit includes a probe specific to the aforementioned gene in the genome that can be used for FISH, and the specific probe consists of the nucleotide sequence of the region from 43.81 Mb to 44.02 Mb on human chromosome 19. The specific probe may be a chain of nucleotide molecules or a combination of two or more (preferably two) chains of nucleotide molecules covering the region from 43.81 Mb to 44.02 Mb on human chromosome 19.

As another assay based on hybridization, comparative genomic hybridization (CGH) can also be used. In CGH, DNA is extracted from each of a test cell to be analyzed and a normal cell to be used as a control, and DNA obtained from each cell is separately labeled with a fluorescent dye of different color. A mixed solution containing equivalent amount of each labeled DNA is prepared and kept at 37° C. for two to three days in the dark to allow test DNA and control DNA to competitively bind to all the sites in chromosomes. As a result, if the copy number of the gene is increased in the test DNA, fluorescence of the color labeled with the test DNA is intensely observed at a position corresponding to the gene on a slide. In contrast, if the copy number of the gene is decreased in the test DNA, fluorescence of the color labeled with the control DNA is intensely observed at a position corresponding to the gene on a slide.

An assay based on amplification includes quantitative nucleic acid amplification. In this method, because the ACTN4 gene in the genome is a template in an amplification reaction, the amplification product is obtained in an amount in proportion to the copy number of the gene. Specific examples of the nucleic acid amplification include quantitative PCR such as quantitative real-time PCR.

In a diagnostic method according to a second aspect of the present invention, whether or not stage I lung adenocarcinoma is poor-prognosis is determined by examining an enhanced expression of α-actinin-4 protein in a stage I lung adenocarcinoma tissue or cell, whereby poor-prognosis stage I lung adenocarcinoma is detected. The above method includes a step of measuring an expression level of ACTN4 gene in the aforementioned stage I lung adenocarcinoma tissue or cell. When the expression level is larger than that in a corresponding normal tissue or cell, that is, an expression of α-actinin-4 protein is enhanced, the stage I lung adenocarcinoma in a subject mammal is determined (diagnosed) as poor-prognosis stage I lung adenocarcinoma.

A publicly known, standardized method in the art can be used to measure an expression level of ACTN4 gene. An expression level of general gene is typically measured using an mRNA or protein level as an index. Similarly in the present invention, an expression level of α-actinin-4 protein can be measured by using an mRNA or protein level of ACTN4. The ACTN4 gene sequence and the sequence of an amino acid encoded by the gene are as described above, and a person skilled in the art can measure the expression level of α-actinin-4 protein using a standardized method in reference to these sequences.

According to a preferred embodiment of the present invention, a step of measuring an expression level of a gene includes measuring an expression level of α-actinin-4 protein in the aforementioned tissue or cell. The expression level of α-actinin-4 protein can be measured by a method known in the art. For example, the amino acid sequence shown in SEQ ID NO: 2 may be given as an amino acid sequence of α-actinin-4 protein, and a person skilled in the art can appropriately measure the amount of the protein in reference to the sequence.

The amount of α-actinin-4 protein can be preferably measured by using, for example, an antibody that binds to the protein. Such an antibody is preferably an antibody specific to α-actinin-4 protein. The antibody may be a polyclonal antibody or a monoclonal antibody. Determination of α-actinin-4 protein is preferably performed by immunohistochemical staining using such an antibody. Alternatively, an immunoblot assay, in which protein extracted from the aforementioned tissue or cell will be subjected to electrophoresis and then determined using an antibody, and the like can be used.

According to another preferred embodiment of the present invention, a step of measuring an expression level of gene includes determining ACTN4 mRNA in the aforementioned tissue or cell. For example, the nucleotide sequence shown in SEQ ID NO: 1 (wherein, t in the sequence is converted to u) may be given as ACTN4 mRNA, and a person skilled in the art can appropriately determine ACTN4 mRNA in reference to the sequence. Specific examples of determination methods include a method in which amplification is performed by RT-PCR using a specific primer pair, followed by electrophoresis.

A normal tissue or cell to be used as a comparative control may be a normal tissue or cell obtained from the identical subject individual or that obtained from a different individual. Also, expression levels of α-actinin-4 protein in normal tissues and cells of a plurality of different individuals are measured and an average value thereof can be used as a comparative control. Further, an appropriate value such as an average value+standard deviation is set in advance based on data of expression levels in a plurality of normal tissues and cells measured in advance, and when the expression level in the test tissue or cell is higher than the appropriate value, it can be determined that the expression of ACTN4 gene is enhanced. Examples of the “corresponding normal tissue or cell” include a normal lung tissue or cell.

In the present specification, it is verified that when a serum CEA concentration of a subject mammal is increased along with an enhanced α-actinin-4 protein expression, the stage I lung adenocarcinoma developed in the subject can be determined as poor-prognosis stage I lung adenocarcinoma with further remarkable significant difference. Therefore, according to a preferred embodiment of the present invention, a diagnostic method according to a second aspect of the present invention includes the following steps of; (c) measuring a concentration of carcinoembryonic antigen (CEA) in serum of the aforementioned mammal, and further, (d) comparing the concentration obtained in (c) with a concentration of carcinoembryonic antigen (CEA) in normal serum. According to the above diagnostic method, when the expression of α-actinin-4 protein in the aforementioned stage I lung adenocarcinoma tissue or cell is enhanced compared to that in the normal tissue or cell, and the concentration of carcinoembryonic antigen (CEA) in the serum of the aforementioned mammal is significantly higher than that in the aforementioned normal serum, it is indicated that the stage I lung adenocarcinoma developed in the mammal is poor-prognosis stage I lung adenocarcinoma.

A publicly known, standardized method in the art can be used as a method for measuring a serum concentration of CEA. CEA is a well-known tumor marker, and various methods for measuring a serum concentration of CEA are known such as ELISA and CLEIA, and a kit for carrying out each of the above methods is commercially available. Normal serum to be used as a comparative example may be normal serum obtained from the identical subject individual, or that obtained from a different individual. Also, concentrations of CEA in normal serum of a plurality of different individuals are measured and an average value thereof can be used as a comparative control. Further, an appropriate value such as an average value+standard deviation is set in advance based on data of concentrations of CEA in a plurality of normal serum measured in advance, and when the concentration in the test serum is higher than the appropriate value, it can be determined that the serum concentration of CEA is increased. For example, in human serum, when a concentration of CEA exceeds 5.0 ng/mL, it can be determined that the concentration is significantly higher than that in normal serum.

According to a preferred embodiment of the present invention, a diagnostic method according to a second aspect of the present invention is performed in combination with a diagnostic method according to a first aspect of the present invention. That is, in this embodiment, a diagnostic method according to a second aspect of the present invention further includes the following step of: (e) measuring the copy number of ACTN4 gene in the genome in a stage I lung adenocarcinoma tissue cell obtained from the aforementioned mammal. According to the above method, when an expression level of α-actinin-4 protein in the aforementioned stage I lung adenocarcinoma tissue or cell is enhanced compared to that in the aforementioned normal tissue or cell, and the copy number of ACTN4 gene exceeds 4, it is indicated that the stage I lung adenocarcinoma developed in the aforementioned mammal is poor-prognosis stage I lung adenocarcinoma.

In order to practice the method of detection/diagnosis according to the present invention as described above, necessary reagents can be collectively provided as a kit. Therefore, according to the present invention, a kit for detecting poor-prognosis stage I lung adenocarcinoma in a mammal is provided, and the kit includes a reagent capable of measuring the copy number of ACTN4 in the genome in a cell. Such a reagent is selected depending on a specific method to be practiced, and it is preferably a probe specific to the gene in the genome that can be used for FISH. The aforementioned specific probe preferably consists of the nucleotide sequence of the region from 43.81 Mb to 44.02 Mb on human chromosome 19.

A kit according to another embodiment of the present invention is a kit for detecting poor-prognosis stage I lung adenocarcinoma in a mammal, including a reagent capable of measuring expression levels of ACTN4 gene and α-actinin-4 protein in a tissue or cell. As such a reagent, a reagent to determine α-actinin-4 protein in the aforementioned tissue or cell, for example, an antibody specifically binding to α-actinin-4 protein, can be preferably used. Alternatively, such a reagent may also be a reagent to determine ACTN4 mRNA in the aforementioned tissue or cell, for example, a specific primer used for RT-PCR using ACTN4 mRNA as a template and another reagent for RT-PCR. According to a preferred embodiment of the present invention, the kit further includes a reagent capable of measuring a serum concentration of carcinoembryonic antigen (CEA). According to another preferred embodiment of the present invention, the kit further includes a reagent capable of measuring the copy number of ACTN4 gene. Such a reagent is preferably a probe specific to the gene in the genome that can be used for FISH. The aforementioned specific probe preferably consists of the nucleotide sequence of the region from 43.81 Mb to 44.02 Mb on human chromosome 19.

The kit of the present invention may further include other reagents, a reaction container, an instruction, and the like, according to a specific method to be practiced.

According to the present invention, a method for treating or preventing stage I lung adenocarcinoma, including administering to a subject a therapeutically effective amount of an ACTN4 inhibitor, is provided. Further, according to the present invention, use of an ACTN4 inhibitor for the production of a medicine for treating stage I lung adenocarcinoma is provided.

A subject to whom treatment or prevention is given is preferably a mammal, for example, a human or a non-human mammal.

According to a preferred embodiment of the present invention, the ACTN4 inhibitor is a small molecule such as a medicine. Such a small molecule is obtainable through screening by a publicly known method in the art, using an ACTN4-inhibitory activity as an index.

According to a preferred embodiment of the present invention, the ACTN4 inhibitor is an antibody specifically binding to α-actinin-4 protein. The antibody can be either a polyclonal antibody or a monoclonal antibody; however, it is preferably a monoclonal antibody.

According to a preferred embodiment of the present invention, the ACTN4 inhibitor is an antisense nucleic acid molecule specifically inhibiting an expression of ACTN4 gene. An antisense method is a known technique to inhibit a specific gene expression. In a specific example, the aforementioned antisense nucleic acid molecule is an approximately 10- to 40-base-long antisense RNA designed based on a sequence of the 5′ coding region of ACTN4 gene. In another specific example, the aforementioned antisense nucleic acid molecule is a DNA oligonucleotide designed to be complementary to a sequence of a region involved in the transcription of the ACTN4 gene. Such antisense nuclei acid molecules can be easily designed based on the ACTN4 gene sequence as shown in SEQ ID NO: 1.

According to a preferred embodiment of the present invention, the ACTN4 inhibitor is a siRNA nucleotide molecule specifically inhibiting an expression of ACTN4 gene. In the present specification, the “siRNA nucleotide molecule” means not only siRNA per se but also a longer double-stranded RNA molecule capable of introducing siRNA into a target cell. The siRNA nucleotide molecule is a known tool capable of inhibiting a specific gene expression by RNA interfering (RNAi) (Elbashir, S. M. et al., Nature 411, 494-498, 2001). Typically, siRNA consists of a nucleotide sequence containing 19 to 21 base pairs homologous to a sequence specific to target gene mRNA. The aforementioned double-stranded RNA molecule typically consists of a longer nucleotide sequence homologous to a sequence specific to target gene mRNA. Such siRNA can be easily designed based on the ACTN4 gene sequence as shown in SEQ ID NO: 1. Further, the aforementioned siRNA nucleotide molecule can also be expressed by an appropriate vector delivered into a cell. Accordingly, the ACTN4 inhibitor may also be a vector expressing a siRNA nucleotide molecule that specifically inhibits an expression of ACTN4 gene. Such a vector can be easily constructed according to a standardized procedure known in the art. (Bass, B. L., Cell 101, 235-238, 2000; Tavernarakis, N. et al., Nat. Genet. 24, 180-183, 2000; Malagon, F. et al., Mol. Gen. Genet. 259, 639-644, 1998; Parrish, S. et al., Mol. Cell. 6, 1077-1087, 2000).

The ACTN4 inhibitor can be administered via a suitable route, for example, locally, intravenously, subcutaneously, intramuscularly, orally, or through rectum or mucous membrane. The therapeutically effective dosage is determined properly by a doctor or veterinarian according to severity of symptoms, age of the patient, efficacy of the specific drug used, administration route, frequency of administration and the like. Generally, the therapeutically effective dosage is approximately 0.001 to approximately 1000 mg/kg body weight, preferably approximately 0.01 to approximately 10 mg/kg body weight, more preferably approximately 0.01 to approximately 1 mg/kg body weight per day.

The ACTN4 inhibitor may be administered together with a pharmaceutically allowable carrier. Thus, the present invention provides a pharmaceutical composition for treatment of stage I lung adenocarcinoma, comprising an ACTN4 inhibitor and a pharmaceutically allowable carrier.

The pharmaceutically allowable carrier, such as vehicle, diluent base or diluent, is selected properly by those who are skilled in the art according to the administration route and the properties of the specific drug used.

The present invention also provides a method for treating stage I lung adenocarcinoma, including a step of administering a therapeutically effective amount of a chemotherapeutic agent to patients with stage I lung adenocarcinoma, who are determined to have poor-prognosis by the diagnostic method in the first aspect or the diagnostic method in the second aspect of the present invention, or by a combination thereof. The chemotherapeutic agent may be the ACTN4 inhibitor described above, a tegafur-5-fluorouracil preparation, another anticancer agent such as cisplatin or the like.

### EXAMPLES

Hereinafter, the present invention will be described more specifically with reference to Examples, which should be understood that the present invention is not restricted thereby.

### Example 1

### Relationship Between Stage I Lung Adenocarcinoma and Acceleration of α-Actinin-4 Gene Expression

The samples used were formalin-fixed paraffin-embedded resected samples from 372 patients, who were subjected to excision surgery of stage I lung adenocarcinoma in the Hospital of National Cancer Center (Japan) during the period from December 1997 to March 2001 (hereinafter, referred to as “cohort 1”). Expression of α-actinin-4 protein in these samples was evaluated by immunohistochemical assay. The primary antibody used in the immunohistochemical assay was anti-human α-actinin-4 rabbit polyclonal antibody prepared in the Research Institute of National Cancer Center (Japan). Immunostaining was carried out by ABC method of using an avidin-biotin complex.

In evaluation of the expression of α-actinin-4 protein, samples in which the expression thereof was accelerated in the cancer region than in the vascular endothelial cells in the same section and the expression-accelerated region occupied 30% or more of the entire tumor area were defined to be positive, and the other sample to be negative.

Then, the classification of α-actinin-4 protein expression and the risk of various clinical pathological factors on the lifetime of patients with stage I lung adenocarcinoma were analyzed by monovariate and multivariate analyses in Cox proportional hazard model. The variables used were, in addition to α-actinin-4 protein expression (positive/negative), sex (male/female), age (65 or older/younger than 65), smoking status (no/yes), stage (IA/IB), serum CEA concentration (more than 5 ng/mL/5 ng/mL or less), lymph duct invasion (positive/negative) and vein invasion (positive/negative). The results are summarized in the following Table 1.

TABLE 1
Analysis of the factors having an influence on clinical pathological outcome of the patients with stage I lung adenocarcinoma
by using Cox proportional hazard model (cohort 1: analysis of α-actinin-4 protein expression)

| | | Monovariate analysis | | | Multivariate analysis | | |
|---|---|---|---|---|---|---|---|
| Variable | N | Hazard rate | 95% Confidence interval | P | Hazard rate | 95% Confidence interval | P |
| Sex | 182/190 | 2.1011 | 1.2388-3.5634 | 0.00588 | 1.581 | 0.8484-2.9462 | 0.149198 |
| (male/female) | | | | | | | |
| Age | 200/172 | 2.5009 | 1.4286-4.3783 | 0.00134 | 2.3074 | 1.3012-4.0916 | 0.00422 |
| (65 or older/younger than 65) | | | | | | | |
| Smoking status | 165/207 | 2.3957 | 1.4203-4.0410 | 0.00106 | 1.7603 | 0.9562-3.2403 | 0.069338 |
| (no/yes) | | | | | | | |
| Stage | 295/77 | 2.6628 | 1.5773-4.4953 | 0.00025 | 1.9322 | 1.1150 -3.3483 | 0.01887 |
| (IA/IB) | | | | | | | |
| Serum CEA | 62/310 | 3.0656 | 1.8055-5.2049 | 3.4E−05 | 2.1566 | 1.2491-3.7237 | 0.00581 |
| (>5 ng/mL/5 ng/mL≧) | | | | | | | |
| Lymph duct invasion | 199/173 | 2.8852 | 1.7313-4.8082 | 4.8E−05 | 2.5643 | 1.5066-4.3646 | 0.00052 |
| (positive/negative) | | | | | | | |
| Vein invasion | 106/263 | 2.5171 | 1.5221-4.1624 | 0.00032 | 1.4718 | 0.8629-2.5105 | 0.156032 |
| (positive/negative) | | | | | | | |
| α-Actinin-4 | 199/173 | 2.1146 | 1.2196-3.6664 | 0.00765 | 2.1153 | 1.1955-3.7425 | 0.01007 |
| protein expression | | | | | | | |
| (positive/negative) | | | | | | | |

The results of monovariate analysis summarized in the left side of Table 1 showed that all of these items could be risk factors which lead to a poor-prognosis.

In addition, the right side of Table 1 summarizes the results obtained by multivariate analysis. The results indicated that age, stage, serum CEA concentration, lymph duct invasion and α-actinin-4 protein expression showed a statistically significant difference, indicating that these items were the risk factors which lead to a poor prognosis. Thus, an intensity of the α-actinin-4 protein expression in stage I lung adenocarcinoma cells was shown to prognosis factor independent of the other clinical pathological factors studied here.

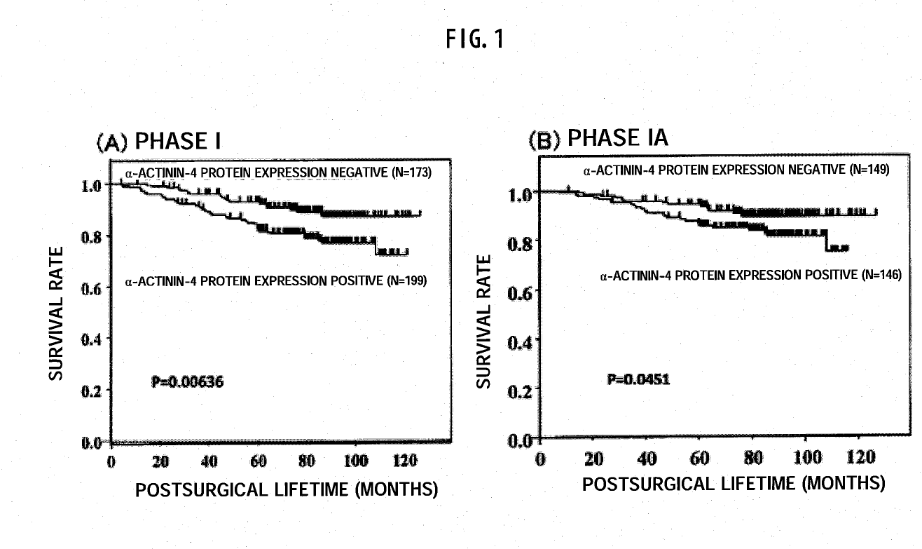

Survival curves of the two groups of incidences, positive and negative cases of α-actinin-4 protein expression, classified as described above were then drawn by the Kaplan-Meier method and examined by the Log-rank test. The survival curves obtained were shown in FIG. 1. Panel A of FIG. 1 shows survival curves of an entire stage I. The 5-year survival rate of the entire stage I in the negative cases was 92.7%, while the 5-year survival rate stage in the positive cases was 82.1%. Panel B of FIG. 1 shows survival curves only of stage IA. The 5-year survival rate of the stage IA in the negative cases was 94.4%, while the 5-year survival rate thereof in the positive cases was 86.5%. According to both panels A and B, the difference in survival rate between the negative and positive cases increases after 5 years from surgery. Thus, FIG. 1 shows that the positive cases of α-actinin-4 protein expression have a poorer prognosis than that of the negative cases with a statistically significant difference.

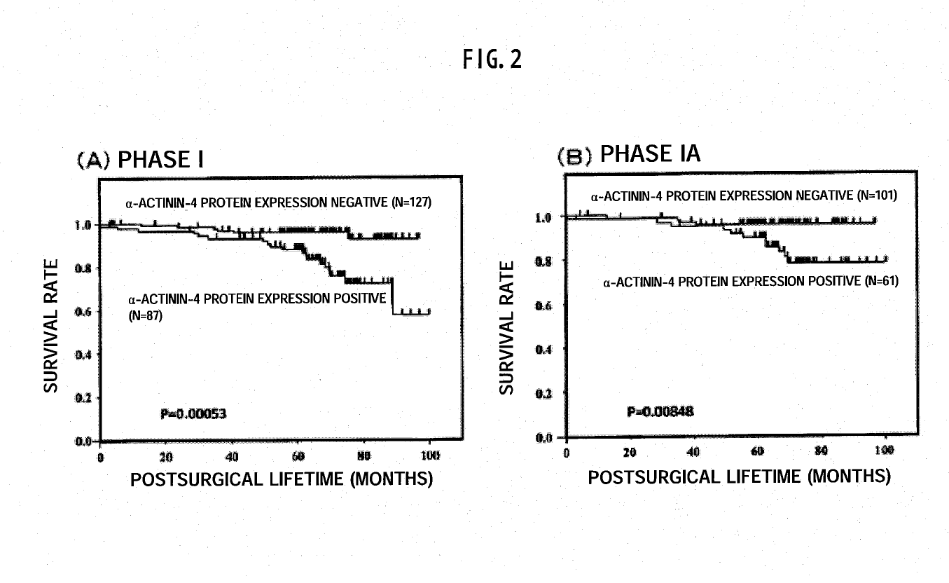

A similar study was also made by using formalin-fixed paraffin-embedded resected samples from 214 patients, who were subjected to excision surgery of stage I lung adenocarcinoma in the Hospital of National Cancer Center (Japan) during the period from March 2001 to January 2003 (hereinafter, referred to as “cohort 2”). The results are summarized in Table 2 and FIG. 2.

TABLE 2
Clinical-pathological analysis of prognostic factors in stage I lung adenocarcinoma by using Cox proportional hazard model
(cohort 2: Analysis of α-actinin-4 protein expression)

| | | Monovariate analysis | | | Multivariate analysis | | |
|---|---|---|---|---|---|---|---|
| Variable | N | Hazard rate | 95% Confidence interval | P | Hazard rate | 95% Confidence interval | P |
| Sex | 100/114 | 0.8792 | 0.3990-1.9376 | 0.749496 | | | |
| (male/female) | | | | | | | |
| Age | 115/99 | 1.8251 | 0.8177-4.0736 | 0.14192 | | | |
| (65 or older/younger than 65) | | | | | | | |
| Smoking status | 120/94 | 0.7797 | 0.3437-1.7690 | 0.55164 | | | |
| (no/yes) | | | | | | | |
| Stage | 162/52 | 2.1994 | 0.9871-4.9004 | 0.053825 | | | |
| (IA/IB) | | | | | | | |
| Serum CEA | 36/178 | 4.2755 | 1.9095-9.5731 | 0.00041 | 3.4228 | 1.4977-7.8222 | 0.00325 |
| (>5 ng/mL/5 ng/mL≧) | | | | | | | |
| Lymph duct invasion | 57/157 | 3.8748 | 1.7566-8.5475 | 0.00079 | 1.9355 | 0.7042-5.3194 | 0.200464 |
| (positive/negative) | | | | | | | |
| Vein invasion | 63/151 | 3.2821 | 1.4871-7.2441 | 0.00326 | 1.7957 | 0.6536-4.9336 | 0.256259 |
| (positive/negative) | | | | | | | |
| α-Actinin-4 | 87/127 | 4.4238 | 1.7654-11.0849 | 0.00151 | 3.5239 | 1.3904-8.9312 | 0.00794 |
| protein expression | | | | | | | |
| (positive/negative) | | | | | | | |

The results obtained by monovariate analysis shown in the left side of Table 2 showed that serum CEA, lymph duct invasion, vein invasion and α-actinin-4 protein expression could be the risk factors which lead to a poor prognosis.

In addition, the right side of Table 2 shows the results obtained by multivariate analysis only of the serum CEA, lymph duct invasion, vein invasion and α-actinin-4 protein expression, which were shown to be factors that lead to a poor prognosis by monovariate analysis. The results showed that serum CEA and α-actinin-4 protein expression showed a statistically significant difference, indicating that these items could be risk factors that lead to a poor prognosis. Thus, the intensity of the α-actinin-4 protein expression in stage I lung adenocarcinoma cells are prognostic factors independent of other clinical pathological factors studied here.

Panel A of FIG. 2 shows survival curves of an entire stage I. The 5-year survival rate of the negative cases in stage I was 95.9%, while the 5-year survival rate of the positive cases in stage I was 87.9%. Panel B of FIG. 2 shows survival curves only of the stage IA. The 5-year survival rate of the negative cases in the stage IA was 95.9%, while the 5-year survival rate of the positive cases in the stage IA was 89.7%. According to both panels A and B, the difference in survival rate between the negative and positive cases increases after 5 years from surgery. Thus, FIG. 2 shows that the positive cases of α-actinin-4 protein expression have a poorer prognosis than the negative cases of α-actinin-4 protein expression with a statistically significant difference.

### Example 2

### Prediction of Prognosis for Stage I Lung Adenocarcinoma by a Combination of Acceleration of α-Actinin-4 Protein Expression and Serum CAE Concentration

The relevance of the combination of acceleration of α-actinin-4 protein expression and serum CEA concentration to a prognosis in the stage I lung adenocarcinoma were studied for the cohorts 1 and 2 described in Example 1. Specifically, the cohorts 1 and 2 were divided into the following three groups: a group of serum CEA of 5 ng/mL or less with negative α-actinin-4 protein; a group of serum CEA of more than 5 ng/mL or α-actinin-4 protein positive; and a group of serum CEA more than 5 ng/mL with positive α-actinin-4 protein, and survival curves of each group were drawn by the Kaplan-Meier method and analyzed by the Log-rank test.

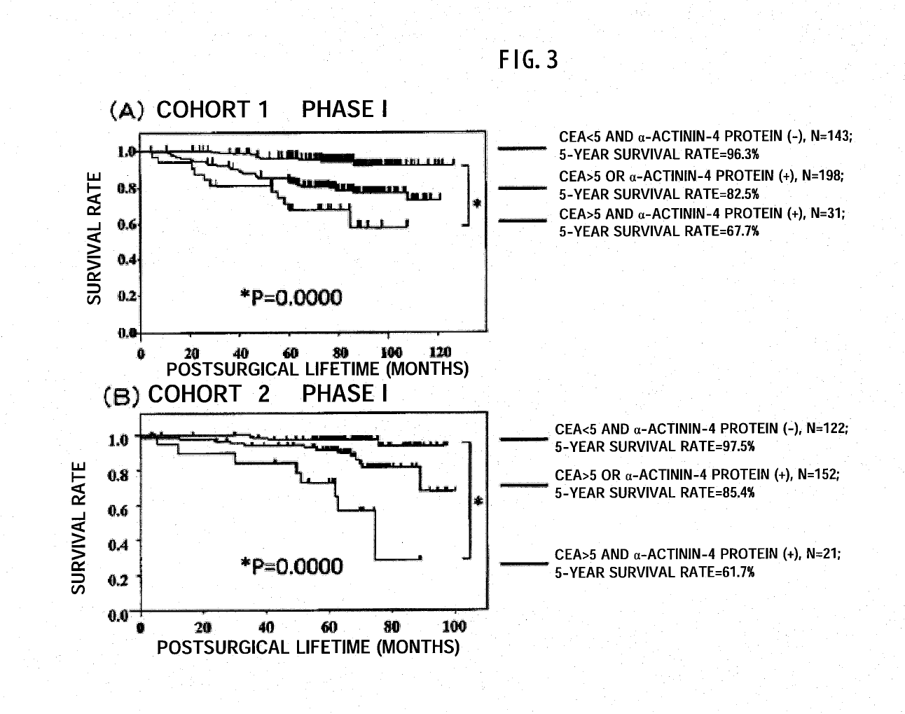

The survival curves obtained are shown in FIG. 3. FIG. 3 shows that the combination of acceleration of α-actinin-4 protein expression and serum CEA concentration is a severer risk factor that lead to a poor prognosis, indicating that the accelerated of α-actinin-4 protein expression in stage I lung adenocarcinoma cells and a serum CEA concentration of more than 5 ng/mL have a distinctively poor postoperative prognosis.

### Example 3

### Increase in Genome Copy Number of ACTN4 Gene in Stage I Lung Adenocarcinoma

A probe specific to the ACTN4 gene present on the long arm of chromosome 19 was prepared to study the copy number of ACTN4 gene on genome, and paraffin sections of dissected stage I lung adenocarcinoma samples were analyzed by the FISH (Fluorescence in Situ Hybridization) method.

The copy number of ACTN4 gene in the paraffin sections of the cohort 1 described in Example 1 was determined by the FISH method. PathVysion DNA probe kit (Abbott Molecular, Des Plaines, Ill.) was used in measurement by the FISH method. The specific probe used in hybridization was a denatured bacterial artificial chromosome (BAC) probe (red) containing the region from 43.81 Mb to 44.02 Mb on human chromosome 19 including ACTN4 (RP11-118P21) gene locus. Chromosome 19 centromere control clone (green) was also used. These probes were labeled by using SpectrimOrange (Abbott Molecular, Des Plaines, Ill.). Hybridization was carried out at 37° C. for 14 to 18 hours. Red and green fluorescence signals in 20 cell nuclei were counted according to the method of Herceptin test, and samples having a value obtained by dividing the red signal count by the green signal count of more than 2 were defined to be ACTN4 amplification (+) (FISH positive) and those having a count of 2 or less to be ACTN4 amplification (−) (FISH negative).

Then, the classification on ACTN4 gene amplification and the risk of various clinical pathological factors on the survival time of stage I lung adenocarcinoma were analyzed by monovariate and multivariate analyses in the Cox proportional hazard model. The variables used were, in addition to the ACTN4 amplification (positive/negative), sex (male/female), age (65 or older/younger than 65), smoking status (no/yes), stage (IA/IB), serum CEA concentration (more than 5 ng/mL/5 ng/mL or less), lymph duct invasion (positive/negative) and vein invasion (positive/negative). The results are summarized in the following Table 3.

TABLE 3
Clinical-pathological analysis of prognostic factors for stagestage I lung adenocarcinoma by using Cox
proportional hazard model (cohort 1: analysis of ACTN4 gene copy number

| | | Monovariate analysis | | | Multivariate analysis | | |
|---|---|---|---|---|---|---|---|
| | | | 95% Confidence | | | 95% Confidence | |
| Variable | N | Hazard rate | interval | P | Hazard rate | interval | P |
| Sex | 98/108 | 2.3951 | 1.1277-5.0868 | 0.02304 | 2.4325 | 1.1437-5.1738 | 0.02097 |
| (male/female) | | | | | | | |
| Age | 86/120 | 2.2737 | 1.0167-5.0848 | 0.04548 | 2.7567 | 1.2133-6.2635 | 0.01545 |
| (65 or older/younger than 65) | | | | | | | |
| Smoking status | 123/83 | 1.8802 | 0.9265-3.8152 | 0.080346 | | | |
| (no/yes) | | | | | | | |
| Stage | 154/52 | 2.3146 | 1.1227-4.7718 | 0.023 | 2.5259 | 1.1998-5.3179 | 0.01471 |
| (IA/IB) | | | | | | | |
| Serum CEA | 34/172 | 1.8416 | 0.8234-4.1189 | 0.137092 | | | |
| (>5 ng/mL/5 ng/mL≧) | | | | | | | |
| Lymph duct invasion | 36/170 | 2.5771 | 1.2117-5.4808 | 0.01394 | 2.8193 | 1.2814-6.2026 | 0.00998 |
| (positive/negative) | | | | | | | |
| Vein invasion | 64/142 | 2.024 | 0.9973-4.1079 | 0.050897 | | | |
| (positive/negative) | | | | | | | |
| ACTN4 gene amplification | 20/186 | 4.2287 | 1.9445-9.1960 | 0.00028 | 3.9933 | 1.7878-8.9195 | 0.00073 |
| (positive/negative) | | | | | | | |

The results of monovariate analysis shown in the left side of Table 3 show that sex, age, stage, lymph duct invasion and ACTN4 amplification could be the risk factors which lead to a poor prognosis.

The right side of Table 3 shows the results obtained by multivariate analysis only of sex, age, stage, lymph duct invasion and ACTN4 amplification which were found to be the potential factors that lead to a poor prognosis in the monovariate analysis above. The results showed that sex, age, stage, lymph duct invasion and ACTN4 amplification showed a statistically significant difference, indicating that these items were risk factors which lead to a poor prognosis. It was thus shown that the ACTN4 amplification in stage I lung adenocarcinoma cells was a prognostic factor independent of the other clinical pathological factors studied here.

### Example 4

### Prediction of Prognosis for Stage I Lung Adenocarcinoma by a Combination of Acceleration of α-Actinin-4 Protein Expression and ACTN4 Gene Amplification

The relevance of the combination of acceleration of α-actinin-4 protein expression and ACTN4 gene amplification to the prognosis in stage I lung adenocarcinoma was studied in the cohort 1 described in Example 1. Specifically, the samples were divided into the following three groups: a group of ACTN4 gene amplification (−) and α-actinin-4 protein expression negative; a group of ACTN4 amplification (−) and actinin-4 expression positive; and a group of ACTN4 gene amplification (+) and α-actinin-4 protein expression positive, and a survival curve of each group was drawn by the Kaplan-Meier method and analyzed by the Log-rank test.

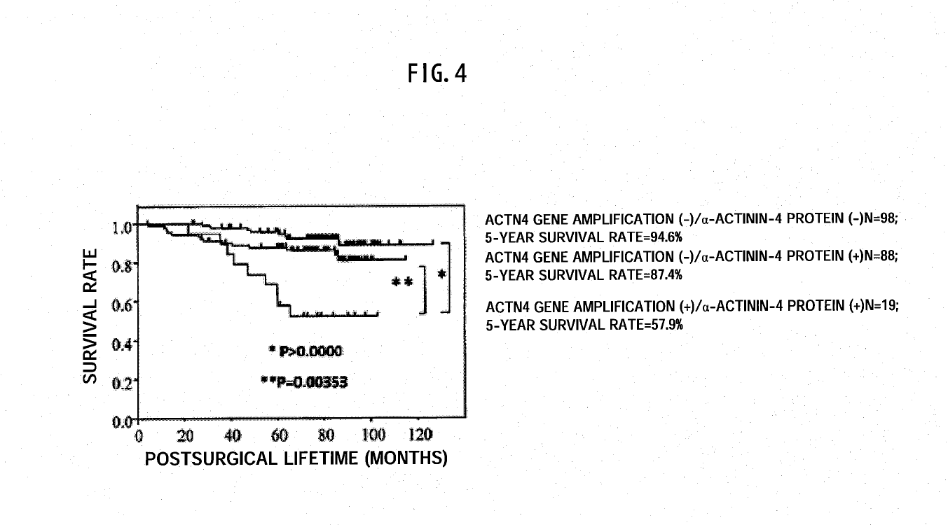

The survival curves obtained were shown in FIG. 4. FIG. 4 shows that the combination of acceleration of α-actinin-4 protein expression and ACTN4 gene amplification is a severer risk factor that lead to a poor prognosis than that of single factor thereof and thus, that patients who exhibit acceleration of α-actinin-4 protein expression and ACTN4 gene amplification in stage I lung adenocarcinoma cells have distinctively poor postoperative prognosis.

### Example 5

### Relationship Between Lung Adenocarcinoma and Acceleration of α-Actinin-4 Gene Expression

A similar study to Example 1 was made and expression of α-actinin-4 protein was evaluated by immunohistochemical assay. The samples used were formalin-fixed paraffin-embedded resected samples (total 540=372+168) from 168 patients, who were subjected to excision surgery of stages II and III lung adenocarcinoma in the Hospital of National Cancer Center (Japan) from 1997 to 2001, in addition to cohort 1 in Example 1 (hereinafter, referred to as “cohort 1”). In addition to cohort 2 described in Example 1, formalin-fixed paraffin-embedded resected samples (total 661=214+447) from 447 patients, who were subjected to excision surgery of stage I lung adenocarcinoma in the Hospital of National Cancer Center East (Japan) from 1993 to 2005 were used (hereinafter, referred to as “cohort 2”). The definitions of the primary antibody used in the immunohistochemical assay, immunostaining method, positive (1HC-positive) and negative (1HC-negative) are the same as those in

### Example 1

An expression of α-actinin-4 protein was analyzed, similar to Example 1, by monovariate analysis and multivariate analysis in Cox proportional hazard model with groups by sex and age, etc., and the hazard rate of each clinical pathological factor on lifetime of patients with lung adenocarcinoma. The results are summarized in the following Table 4.

TABLE 4
Hazard Ratios for Death in 540 Cases of Cohort 1 (All Stages), According to Prognostic Factors

| | | Univariate analysis | | | Multivariate analysis | | |
|---|---|---|---|---|---|---|---|
| Characteristics | | Hazard Ratio | [95% CI¶] | P Value* | Hazard Ratio | [95% CI¶] | P Value* |
| Sex | (Female vs. Male) | 1.37 | [1.00-1.86] | 0.05 | 1.06 | [0.74-1.51] | 0.76 |
| Age | (<65 yr vs. ≧65 yr) | 1.14 | [0.84-1.55] | 0.38 | | | |
| Smoking history | (Never smoked vs. Current | 1.49 | [1.09-2.02] | 0.01 | 1.06 | [0.74-1.52] | 0.76 |
| | or former | | | | | | |
| Serum CEA¶ level | (≦5 ng/ml vs. >5 ng/ml) | 2.40 | [1.75-3.30] | <0.001 | 1.50 | [1.08-2.08] | 0.02 |
| Pathological stage† | (I vs. II or III) | 5.68 | [4.13-7.81] | <0.001 | 4.01 | [2.78-5.77] | <0.001 |
| Histological | (Well differentiated§ vs. | 3.19 | [2.32-4.41] | <0.001 | 1.57 | [1.09-2.26] | 0.02 |
| differentiation | Moderately or | | | | | | |
| | poorly differentiated) | | | | | | |
| Actinin-4 protein | (Negative vs. Positive) | 1.86 | [1.33-2.58] | <0.001 | 1.64 | [1.17-2.29] | 0.004 |

¶Abbreviations: CEA, carcinoembryonic antigen; Cl, confidence interval.
*Cox regression analysis P values of <0.05 are shown in bold.
†According to the International Union Against Cancer (UICC) TNM Classification of Malignant Tumours, 6th edition (2002).
§Includes bronchioloalveolar carcinoma [World Health Organization (WHO) Histological Typing of Lung and Pleural Tumours, 3rd edition (1995)].

The results of monovariate analysis summarized in the left side of Table 4 show that all of these items above could be risk factors which lead to a poor-prognosis.

Further, the right side of Table 4 summarizes the results obtained by the multivariate analysis. The results indicate that stage, serum CEA concentration, and α-actinin-4 protein expression showed a statistically significant difference, indicating that these items were the risk factors which lead to a poor prognosis. Thus, the intensity of the α-actinin-4 protein expression in stages I-III lung adenocarcinoma cells was shown to be a prognostic factor independent of the other clinical pathological factors studied here.

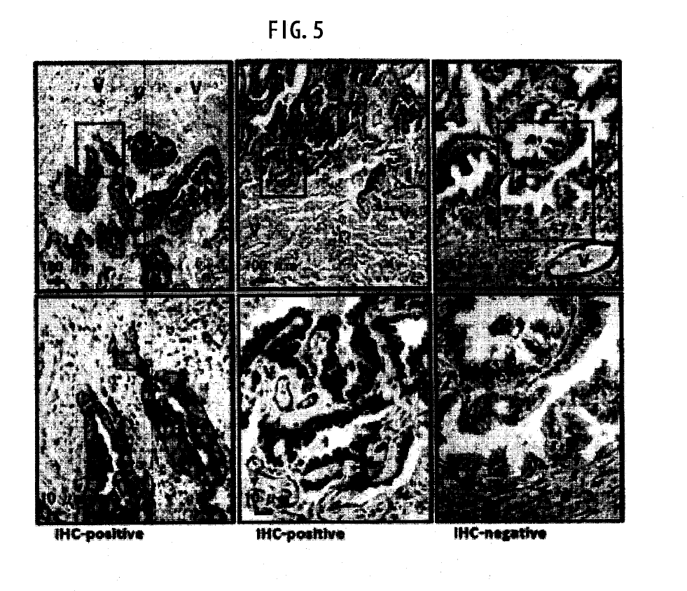

FIG. 5 shows photo-micrographs of lung adenocarcinoma tissue from the two groups divided as above, the positive cases and the negative cases of α-actinin-4 protein expression. The left side and in the middle of FIG. 5 show the results for the positive cases (IHC-positive), while the right side in FIG. 5 shows the results for the negative cases (IHC-negative). The photo-micrographs in the lower side of FIG. 5 show magnified within-frame photos of photo-micrographs that are shown in the upper side thereof. As shown in the figure, actinin-4 protein (“V” in the figure) was extended along alveolar septum, and was localized in cell membrane and cytoplasm of cancer cells invading through interfibrillar mass.

Next, the survival curves for the two groups, the positive cases and the negative cases of α-actinin-4 protein expression were prepared by the Kaplan-Meier method and examined by the Log-rank test. The survival curves obtained are shown in FIG. 6. Panel A of FIG. 6 shows survival curves in stages I-III. The 5-year survival rate of the negative cases in the stages I-III was 82%, while the 5-year survival rate of the positive cases was 69%. Panel B of FIG. 6 shows the survival curves in stage I. The 5-year survival rate of the negative cases in the stage I was 92%, while the 5-year survival rate of the positive cases was 84%. According to both panels A and B, the differences in the survival rates between the negative and positive cases increases after 5 years from surgery. Thus, FIG. 6 shows that the positive cases of α-actinin-4 protein expression have a poorer prognosis than that of the negative cases with a statistically significant difference.

### Example 6

### Increase in Genome Copy Number of ACTN4 Gene in Patients with Stage I Lung Adenocarcinoma

A probe specific to the ACTN4 gene present on the long arm of chromosome 19 was prepared to study the copy number of ACTN4 gene on genome, and paraffin sections of dissected stage I lung adenocarcinoma samples were analyzed using a FISH (Fluorescence in Situ Hybridization) method, similar to that of Example 3. The samples used were formalin-fixed paraffin-embedded resected samples from 543 patients, who were subjected to excision surgery of stages I-IV lung adenocarcinoma in the Hospital of National Cancer Center (Japan) from 1997 to 2009. Of 543 samples, fibroblast growth factor receptor (FGFR) gene mutation was present in 288 samples, while no mutation was present in 255 samples. These 543 samples do not overlap with cohorts 1 and 2 described in Example 5. Similar definitions to those used in Example 3 for the probe specific, positive (FISH-positive) and negative (FISH-negative), are used.

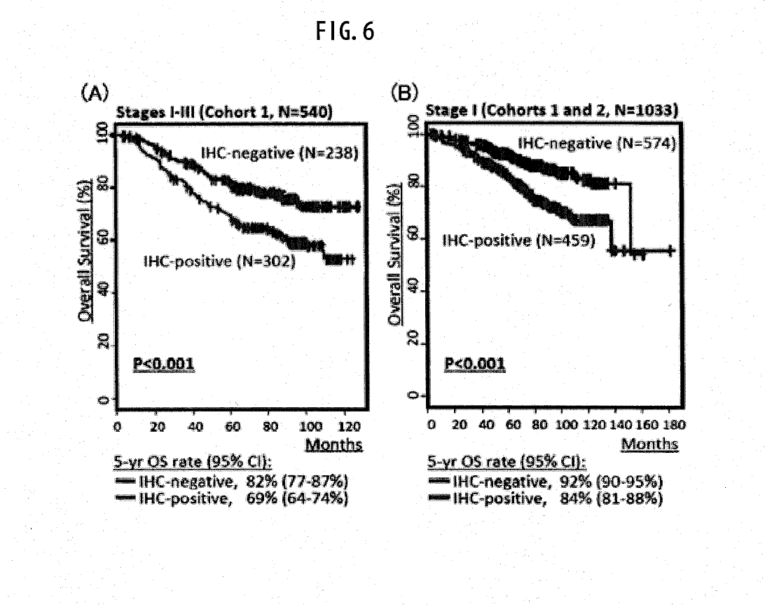

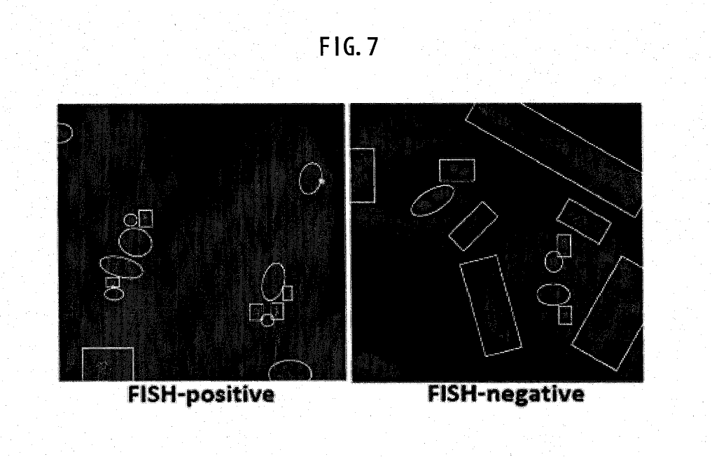

FIG. 7 shows photo-micrographs of lung adenocarcinoma cells signaled by the Fluorescence in Situ Hybridization (FISH) method. The left side in FIG. 7 shows the results for positive cases (FISH-positive), while the right side in FIG. 7 shows the results for negative cases (FISH-negative). As shown in the figure, red (∘ in FIG. 7) and green (□ in FIG. 7) fluorescence signals (fluorescence signals) in lung adenocarcinoma cells were acquired. The FISH positive 79 resected samples (15%) out of 543 resected samples in stages I-IV were detected according to the definitions of FISH positive and negative.

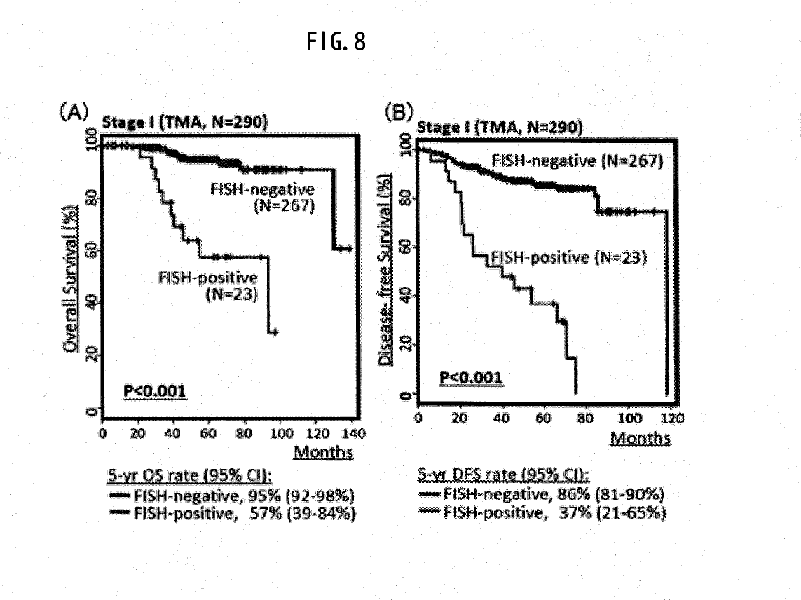

Further, survival curves were drawn by the Kaplan-Meier method for the two groups divided by the FISH method, the positive cases and the negative cases, and were analyzed by the Log-rank test. The obtained survival curves are shown in FIG. 8. Panel A of FIG. 8 shows the survival curves in the stage I. The 5-year survival rate of the negative cases in the stage I was 95%, while the 5-year survival rate thereof in the positive cases was 57%. Panel B of FIG. 8 shows the survival curves in the stage I. The 5-year survival rate of the stage I patients in the negative group was 86%, while the 5-year survival rate thereof in the positive cases was 37%. According to the both Panels A and B, the differences in survival rates between the negative and positive cases increase after 5 years from surgery. Thus, FIG. 8 shows that the positive cases of α-actinin-4 protein expression have a poorer prognosis than that of the negative cases with a statistically significant difference.

### Example 7

### Combination of Immunohistochemical Assay and Fish Method

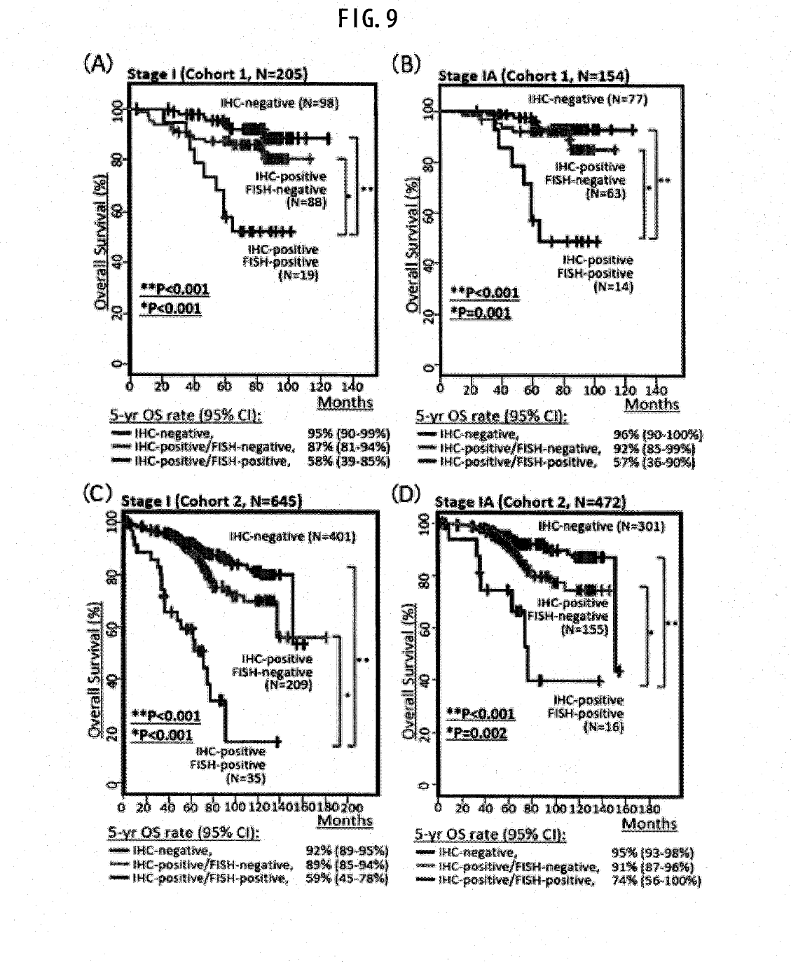

The samples used were 206 resection samples of stage I lung adenocarcinoma from cohort 1 used in Example 5, and 645 resection samples of stage I lung adenocarcinoma from cohort 2 used in Example 5. The negative and positive cases of α-actinin-4 protein expression were combined respectively with the positive and negative cases of the FISH method analysis, and the survival curves of these samples were drawn by the Kaplan-Meier method, then analyzed by the Log-rank test. The obtained survival curves are shown in FIG. 9. Panel A of FIG. 9 shows the survival curves of cohort 1 in the stage I. The 5-year survival rate of the negative cases of α-actinin-4 protein expression in the stage I was 95%, while the positive cases of α-actinin-4 protein expression with the negative cases of the FISH method analysis was 87%. On the other hand, the 5-year survival rate of the positive cases of α-actinin-4 protein expression with the positive cases of the FISH method analysis was 58%. Further, Panel B of FIG. 9 shows the survival curves of cohort 1 in the stage IA. The 5-year survival rate of the negative cases of α-actinin-4 protein expression was 96%, while the positive cases of α-actinin-4 protein expression with the negative cases of the FISH method analysis was 92%. On the other hand, the 5-year survival rate of the positive cases of α-actinin-4 protein expression and with the positive cases of the FISH method analysis was 57%. Thus, Panels A and B show that the positive/negative cases of the FISH method analysis have a poorer prognosis than that of the positive/negative cases of α-actinin-4 protein expression with a statistically significant difference.

Further, Panel C of FIG. 9 shows the survival curves of cohort 2 in the stage I. The 5-year survival rate of the negative cases of α-actinin-4 protein expression was 92%, while the positive cases of α-actinin-4 protein expression with the negative cases of the FISH method analysis was 89%. On the other hand, the 5-year survival rate of the positive cases of α-actinin-4 protein expression with the positive cases of the FISH method analysis was 59%. Further, Panel D of FIG. 9 shows the survival curves of cohort 2 in the stage IA. The 5-year survival rate of the negative cases of α-actinin-4 protein expression was 95%, while the 5-year survival rate of the positive cases of α-actinin-4 protein expression with the negative cases of α-actinin-4 protein expression was 91%. On the other hand, the 5-year survival rate of the positive cases of α-actinin-4 protein expression with the positive cases of the FISH method analysis was 74%. Thus, Panels C and D show that the positive/negative cases have a poorer prognosis than that of the positive/negative cases of α-actinin-4 protein expression with a statistically significant difference.

It should be understood that the present invention is not intended to be limited to the modifications and the preferred embodiments contained herein. Various changes and modifications apparent to those skilled in the art are deemed to be within the spirit and scope of the present invention.

This application is based on Japanese Patent Application No. 2010-78772, filed on Mar. 30, 2010, and incorporates the specification, scope of claims, and drawings thereof. The entire disclosure of the above Japanese Patent Application is incorporated herein by reference.

### INDUSTRIAL APPLICABILITY

The present invention, a method of predicting a prognosis of lung adenocarcinoma and a kit for detecting a lung adenocarcinoma, can be utilized in predicting a prognosis of a stage I lung adenocarcinoma and selecting an appropriate treatment. A pharmaceutical composition of the present invention for treating an adenocarcinoma can be used in treatment of a poor-prognosis stage I lung adenocarcinoma.

1. A method of predicting a prognosis by detecting a poor-prognosis stage I lung adenocarcinoma in a mammal, comprising
a step of measuring the copy number of α-actinin-4 gene (ACTN4) in a stage I lung adenocarcinoma tissue cell obtained from the mammal, wherein,
when the copy number of said gene exceeds 4, the stage I lung adenocarcinoma in the mammal is predicted to be a poor-prognosis stage I lung adenocarcinoma.

2. The method according to claim 1, wherein the copy number of said gene is measured by a FISH method using a probe specific for the gene on genome.

3. The method according to claim 2, wherein said specific probe has the nucleotide sequence of the region from 43.81 Mb to 44.02 Mb on human chromosome 19.

4. A kit for detecting a poor-prognosis stage I lung adenocarcinoma in a mammal, comprising a reagent that is available for determining the copy number of ACTN4 gene in a cell.

5. The kit according to claim 4, wherein said reagent is a probe specific for the gene on genome.

6. The kit according to claim 5, wherein said specific probe has the nucleotide sequence of the region from 43.81 Mb to 44.02 Mb on human chromosome 19.

7. A method of predicting a prognosis by detecting a poor-prognosis stage I lung adenocarcinoma in a mammal, comprising
(a) a step of measuring an expression level of ACTN4 gene in the tissue or the cell of stage I lung adenocarcinoma obtained from said mammal and
(b) a step of comparing the expression level obtained in the step (a) with the expression level of ACTN4 gene in the corresponding normal tissue or cell, wherein,
when gene expression is enhanced greater in said tissue or cell of the stage I lung adenocarcinoma than that of said normal tissue or cell, the stage I lung adenocarcinoma in said mammal is predicted to be a poor-prognosis stage I lung adenocarcinoma.

8. The method according to claim 7, wherein α-actinin-4 protein in said tissue or cell is detected in the step (a).

9. The method according to claim 8, wherein α-actinin-4 protein has the amino acid sequence represented by SEQ ID No. 2.

10. The method according to claim 8, wherein the α-actinin-4 protein is detected by immunohistochemical staining using a specific antibody.

11. The method according to claim 7, wherein the mRNA of α-actinin-4 in said tissue or cell is detected in the step (a).

12. The method according to claim 11, wherein the mRNA of α-actinin-4 has the nucleotide sequence represented by SEQ ID No. 1 (wherein, t in the sequence is replaced with u).

13. The method according to claim 7, further comprising the following steps:
(c) a step of measuring the concentration of carcinoembryonic antigen (CEA) in the serum obtained from said mammal and
(d) a step of comparing the concentration obtained in the step (c) with the concentration of the carcinoembryonic antigen (CEA) in normal serum, wherein,
when expression of the gene is enhanced greater in said tissue or cell of stage I lung adenocarcinoma than that of said normal tissue or cell, and the concentration of the carcinoembryonic antigen (CEA) in the serum obtained from said mammal is statistically significantly higher than that in said normal serum, said stage I lung adenocarcinoma of the mammal is predicted to be a poor-prognosis stage I lung adenocarcinoma.

14. The method according to claim 13, wherein, when the concentration obtained in said step (c) exceeds 5.0 ng/mL, it is determined to be statistically significantly higher than that in normal serum.

15. The method according to claim 7, further comprising the following step:
(e) A step of measuring the copy number of ACTN4 gene in cell of the tissue of stage I lung adenocarcinoma obtained from said mammal, wherein, when expression of the gene is enhanced greater in said tissue or cell of stage I lung adenocarcinoma than that of said normal tissue or cell, and the copy number of said gene exceeds 4, said stage I lung adenocarcinoma of the mammal is predicted to be a poor-prognosis stage I lung adenocarcinoma.

16. The method according to claim 15, wherein said copy number of said gene is determined by a FISH method using a probe specific for the gene on genome.

17. The method according to claim 16, wherein said specific probe has the nucleotide sequence of the region from 43.81 Mb to 44.02 Mb on human chromosome

18. A kit for detecting a poor-prognosis stage I lung adenocarcinoma in a mammal, comprising a reagent that can measure the expression level of ACTN4 gene in the tissue or cell.

19. The kit according to claim 18, wherein said reagent is a reagent for quantitative determination of α-actinin-4 protein in said tissue or cell.

20. The kit according to claim 19, wherein the reagent for quantitative determination of α-actinin-4 protein is an antibody specifically binding to α-actinin-4 protein.

21. The kit according to claim 18, wherein said reagent is a reagent for quantitative determination of the mRNA of α-actinin-4 in the tissue or cell.

22. The kit according to claim 18, further comprising a reagent that can measure the concentration of carcinoembryonic antigen (CEA) in serum.

23. The kit according to claim 18, further comprising a reagent that can measure the copy number of ACTN4 gene in cells.

24. The kit according to claim 23, wherein said reagent is a probe specific for said gene on genome.

25. The kit according to claim 24, wherein said specific probe has the nucleotide sequence of the region from 43.81 Mb to 44.02 Mb on human chromosome 19.

26. A pharmaceutical composition for treating a lung adenocarcinoma, comprising an α-actinin-4 inhibitor and a pharmaceutically acceptable carrier.

27. The pharmaceutical composition according to claim 26, wherein the α-actinin-4 inhibitor is a small molecule.

28. The pharmaceutical composition according to claim 26, wherein the α-actinin-4 inhibitor is an antibody specifically binding to α-actinin-4 protein.

29. The pharmaceutical composition according to claim 26, wherein the α-actinin-4 inhibitor is an antisense nucleotide molecule specifically inhibiting an expression of the ACTN4 gene.

30. The pharmaceutical composition according to claim 26, wherein the α-actinin-4 inhibitor is a siRNA nucleotide molecule specifically inhibiting an expression of the ACTN4 gene.

31. The pharmaceutical composition according to claim 26, wherein the α-actinin-4 inhibitor is a vector expressing the siRNA nucleotide molecule specifically inhibiting an expression of the ACTN4 gene.

32. A kit for measuring the copy number of ACTN4 gene in human cells, comprising a probe specific for the gene on genome that can be used in a FISH method, wherein the specific probe has the nucleotide sequence of the region from 43.81 Mb to 44.02 Mb on human chromosome 19.


SEQUENCE LISTING (2 sequences)

SEQ ID NO: 1 - DNA, 3893 bp, Homo sapiens
  CDS (60)..(2792)
agaggggcgggagctgaggcgggagcggacaggctggtgggcgagcgagaggcggcggaatggtggactaccacgcggcgaaccagtcgtaccagtacggccccagcagcgcgggcaatggcgctggcggcgggggcagcatgggcgactacatggcccaggaggacgactgggaccgggacctgctgctggacccggcctgggagaagcagcagcgcaagaccttcacggcatggtgcaactcccacctgcggaaggcaggcacacagatcgagaacattgatgaggacttccgagacgggctcaagctcatgctgctcctggaggtcatatcaggggagcggttacctaagccggagcgggggaagatgagagtgcacaaaatcaacaatgtgaacaaagcgctggactttattgccagcaaaggcgtcaagctggtctccatcggggcagaagagattgtggacggcaacgcaaagatgaccctgggaatgatctggaccatcatccttaggttcgccatccaggacatctccgtggaagagacctcggccaaggaagggctccttctctggtgccagagaaagacagccccgtataagaacgtcaatgtgcagaacttccacatcagctggaaggatggtcttgccttcaatgccctgatccaccggcacagaccagagctgattgagtatgacaagctgaggaaggacgaccctgtcaccaacctgaacaatgccttcgaagtggctgagaaatacctcgacatccccaagatgctggatgcagaggacatcgtgaacacggcccggcccgacgagaaggccataatgacctatgtgtccagcttctaccatgccttttcaggagcgcagaaggctgaaactgccgccaaccggatctgtaaggtgctggctgtcaaccaagagaacgagcacctgatggaggactacgagaagctggccagcgacctcctggagtggatccggcgcaccatcccctggctggaggaccgtgtgccccaaaagactatccaggagatgcagcagaagctggaggacttccgcgactaccggcgtgtgcacaagccgcccaaggtgcaggagaagtgccagctggagatcaacttcaacacgctgcagaccaagctgcgcctcagcaaccggcccgccttcatgccctccgagggcaagatggtctcggacatcaacaatggctggcagcacttggagcaggctgagaagggctacgaggagtggctgctgaatgagatccgcaggctggagcggctcgaccacctggcagagaagttccggcagaaggcctccatccacgaggcctggactgacgggaaggaagccatgctgaagcaccgggactacgagacggccacactatcggacatcaaagccctcattcgcaagcacgaggccttcgagagcgacctggctgcgcaccaggaccgcgtggagcagatcgccgccattgcccaggagctcaacgagctggattactacgactcccacaatgtcaacacccggtgccagaagatctgtgaccagtgggacgccctcggctctctgacacatagtcgcagggaagccctggagaaaacagagaagcagctggaggccatcgaccagctgcacctggaatacgccaagcgcgcggcccccttcaacaactggatggagagcgccatggaggacctccaggacatgttcatcgtccataccatcgaggagattgagggcctgatctcagcccatgaccagttcaagtccaccctgccggacgccgatagggagcgcgaggccatcctggccatccacaaggaggcccagaggatcgctgagagcaaccacatcaagctgtcgggcagcaacccctacaccaccgtcaccccgcaaatcatcaactccaagtgggagaaggtgcagcagctggtgccaaaacgggaccatgccctcctggaggagcagagcaagcagcagtccaacgagcacctgcgccgccagttcgccagccaggccaatgttgtggggccctggatccagaccaagatggaggagatcgggcgcatctccattgagatgaacgggaccctggaggaccagctgagccacctgaagcagtatgaacgcagcatcgtggactacaagcccaacctggacctgctggagcagcagcaccagctcatccaggaggccctcatcttcgacaacaagcacaccaactataccatggagcacatccgcgtgggctgggagcagctgctcaccaccattgcccgcaccatcaacgaggtggagaaccagatcctcacccgcgacgccaagggcatcagccaggagcagatgcaggagttccgggcgtccttcaaccacttcgacaaggatcatggcggggcgctggggcccgaggagttcaaggcctgcctcatcagcctgggctacgacgtggagaacgaccggcagggtgaggccgagttcaaccgcatcatgagcctggtcgaccccaaccatagcggccttgtgaccttccaagccttcatcgacttcatgtcgcgggagaccaccgacacggacacggctgaccaggtcatcgcttccttcaaggtcttagcaggggacaagaacttcatcacagctgaggagctgcggagagagctgccccccgaccaggccgagtactgcatcgcccgcatggcgccataccagggccctgacgccgtgcccggtgccctcgactacaagtccttctccacggccttgtatggcgagagcgacctgtgaggccccagagacctgacccaacacccccgacggcctccaggaggggcctgggcagccccacagtcccattcctccactctgtatctatgcaaagcactctctgcagtcctccggggtgggtgggtgggcagggaggggctggggcaggctctctcctctctctctttgtgggttggccaggaggttcccccgaccaggttggggagacttggggccagcgcttctggtctggtaaatatgtatgatgtgttgtgcttttttaaccaaggaggggccagtggattcccacagcacaaccggtcccttccatgccctgggatgcctcaccacacccaggtctcttcctttgctctgaggtcccttcaaggcctccccaatccaggccaaagccccatgtgccttgtccaggaactgcctgggccatgcgaggggccagcagagggcgccaccaccacctgacggctggggacccacccagcccctctcccctctctgctccagactcacttgccattgccaggagatggccccaacaagcaccccgcttttgcagcagaggagctgagttggcagaccgggcccccctgaaccgcaccccatcccaccagccccggccttgctttgtctggcctcacgtgtctcagattttctaagaaccaaaaaaaaaaaaggaaaaaaaacacaaaacaacaaaaaccaaaaaaaaaaaaaatcacaaaaacaaaaaaactataaaaaagaaagaattaaaaactttcagagaattactatttactttattaacttacggatttattatataaatatatattcacctagcaacatatctctgccgtctctcctgctctcataatgaagacatagccgattctctgcccgggccccttgctgatgctcctccgggtctgcgtcgggcgtgggtctctggggaccctccagaggtggaggtgggctgatggcctggctgcctggtggttgatggttttgctccccctaccttttttttttgagtttattctgattgattttttttcttggtttctggataaaccaccctctggggacaggataataaaacatgtaatatttttaagaagg

SEQ ID NO: 2 - protein, 911 aa, Homo sapiens
MVDYHAANQSYQYGPSSAGNGAGGGGSMGDYMAQEDDWDRDLLLDPAWEKQQRKTFTAWCNSHLRKAGTQIENIDEDFRDGLKLMLLLEVISGERLPKPERGKMRVHKINNVNKALDFIASKGVKLVSIGAEEIVDGNAKMTLGMIWTIILRFAIQDISVEETSAKEGLLLWCQRKTAPYKNVNVQNFHISWKDGLAFNALIHRHRPELIEYDKLRKDDPVTNLNNAFEVAEKYLDIPKMLDAEDIVNTARPDEKAIMTYVSSFYHAFSGAQKAETAANRICKVLAVNQENEHLMEDYEKLASDLLEWIRRTIPWLEDRVPQKTIQEMQQKLEDFRDYRRVHKPPKVQEKCQLEINFNTLQTKLRLSNRPAFMPSEGKMVSDINNGWQHLEQAEKGYEEWLLNEIRRLERLDHLAEKFRQKASIHEAWTDGKEAMLKHRDYETATLSDIKALIRKHEAFESDLAAHQDRVEQIAAIAQELNELDYYDSHNVNTRCQKICDQWDALGSLTHSRREALEKTEKQLEAIDQLHLEYAKRAAPFNNWMESAMEDLQDMFIVHTIEEIEGLISAHDQFKSTLPDADREREAILAIHKEAQRIAESNHIKLSGSNPYTTVTPQIINSKWEKVQQLVPKRDHALLEEQSKQQSNEHLRRQFASQANVVGPWIQTKMEEIGRISIEMNGTLEDQLSHLKQYERSIVDYKPNLDLLEQQHQLIQEALIFDNKHTNYTMEHIRVGWEQLLTTIARTINEVENQILTRDAKGISQEQMQEFRASFNHFDKDHGGALGPEEFKACLISLGYDVENDRQGEAEFNRIMSLVDPNHSGLVTFQAFIDFMSRETTDTDTADQVIASFKVLAGDKNFITAEELRRELPPDQAEYCIARMAPYQGPDAVPGALDYKSFSTALYGESDL